(19) United States
(12) Patent Application Publication
(10) Pub. No.: US 2005/0049174 A1
(43) Pub. Date: Mar. 3, 2005

(54) Method and substance for facilitating weaning, reducing morbidity and reducing mortality in cardiac surgeries involving extra-corporal circulation

(76) Inventor: Andre Denault, Longueuil (CA)

Correspondence Address: Louis Tessier, P.O. Box 54029, Town of Mount-Royal, QC, H3P 3H4, CA

(21) Appl. No.: 10/926,973

(22) Filed: Aug. 27, 2004

Related U.S. Application Data
(60) Provisional application No. 60/498,360, filed on Aug. 28, 2003.
(60) Provisional application No. 60/498,607, filed on Aug. 29, 2003.
(60) Provisional application No. 60/498,608, filed on Aug. 29, 2003.
(60) Provisional application No. 60/498,359, filed on Aug. 28, 2003.

(51) Int. Cl. A61K 31/00

(52) U.S. Cl. 514/1; 604/500

(57) ABSTRACT

Prophylactic strategies aimed at delivering vasodilators through inhalation in the pulmonary tree treat and prevent right ventricular dysfunction by reducing right ventricular afterload, facilitate separation from bypass and consequently decrease hemodynamic complications, morbidity and mortality. Examples of suitable vasodilatator include prostacyclin (flolan®), amrinone (inocor®), dobutamine (dobutrex®), nitroglycerine, nitroprussiate (nipruss®) and milrinone (primacor®).

This application claims priority from U.S. Provisional Patent Application Ser. No. 60/498,360 filed Aug. 28, 2003, Ser. No. 60/498,607 filed Aug. 29, 2003, Ser. No. 60/498,608 filed Aug. 29, 2003, and Ser. No. 60/498,359 filed Aug. 28, 2003.

### FIELD OF THE INVENTION

The present invention relates to a method and substance for facilitating weaning and reducing morbidity and mortality of subjects undergoing cardiac surgery involving extra-corporal circulation. Specifically, the present invention concerns the use of a vasodilatator, such as milrinone and prostacyclin, administered through the airways of the subject to lessen the chances that the subject experiences a difficult separation from the extra-corporal circulation.

### BACKGROUND OF THE INVENTION

Medical Context

The major cause of death after cardiac surgery is hemodynamic instability. There are specific factors that can predispose a patient to hemodynamic instability. These factors are related to the inability of the heart to relax and accept or receive blood, which is called diastolic dysfunction. When the heart experiences diastolic dysfunction, it requires a higher pressure to be filled, which in some cases leads to serious problem such as pulmonary edema or cardiac malfunction. The latter manifests itself as hemodynamic instability that can lead to death.

There are several types and causes of hemodynamic instability that can occur alone or in combination⁵¹. A few are presented hereinbelow:

Reduced Left and Right Ventricular Contractility, Caused by:
Myocardial ischemia related complication (intra or extracardiac rupture, reduced function);
Intraoperative coronary occlusion (air, clot, calcium);
Coronary graft malfunction (vascular spasm);
Myocardial depression from extra-cardiac factors (brain injury, sepsis); and
Suboptimal cardioplegia.

Increased Left and Right Ventricular Afterload, Caused by:
Primary or secondary pulmonary hypertension;
Left ventricular outflow tract obstruction (after mitral repair or aortic surgery; presence of left ventricular hypertrophy);
Acute aortic dissection from the aortic canulation; and
Right outflow ventricular tract obstruction (mechanical in off-pump bypass surgery or dynamic with right ventricular hypertrophy);
Pulmonary embolism (air, clot, carbon dioxide); and
Hypoxia from pulmonary edema or from right-to-left shunt due to patent foramen ovale.

Abnormal Left and Right Ventricular Filling:
Myocardial left and right ventricular diastolic dysfunction;
Abnormal left ventricular filling from right ventricular dilatation or pulmonary hypertension; and
Extra-cardiac limitation to cardiac filling (pericardial tamponade, positive-pressure ventilation, thoracic tamponade, abdominal compartment syndrome).

Reduced Preload:
Reduced systemic vascular resistance (drugs, sepsis, hemodilution, anaphylaxis); and
Blood losses (external, thoracic, gastrointestinal, retroperitoneal).

Valvular Insufficiency:
Mitral valve insufficiency from ischemia, LVOT obstruction, sub-optimal repair, complication of aortic valve surgery;
Aortic valve insufficiency after mitral valve surgery, dysfunctional prosthesis, aortic dissection; and
Tricuspid valve insufficiency from right ventricular failure.

Costachescu et al⁵¹ documented that diastolic dysfunction was the most common echocardiographic abnormality in hemodynamically unstable patients. Interestingly, right ventricular filling abnormalities were more common than left ventricular filling abnormalities. Right ventricular diastolic dysfunction can be diagnosed using both hemodynamic and echocardiographic criteria. The hemodynamic criteria are obtained through continuous monitoring of the right ventricular pressure waveform and the echocardiographic criteria from the analysis of trans-tricuspid blood flow, hepatic venous flow and interrogation of the tricuspid annulus using tissue Doppler.

In addition, it was observed that the problem with the filling of the right ventricle is a direct consequence of the elevated pressure and is worse on the right side of the heart. Consequently, by reducing the pressure of the heart, particularly on the right side, diastolic dysfunction may be prevented and hemodynamic instability and death thereby avoided. However few drugs can reduce the cardiac pressure on the right side without also reducing the pressure also in the systemic arterial pressure.

Definitions

Unless otherwise defined, the terms of art appearing in this document have the meanings that are understood by those skilled in the art. While many of the terms used in this text do not have a standardized signification, the following definitions will be used throughout this document:
CPB: cardiopulmonary bypass;
DSB=difficult separation from bypass defined as a systolic blood pressure below 80 mm Hg confirmed with central measurement (femoral or aortic), diastolic pulmonary artery pressure or pulmonary artery capillary wedge pressure >15 mm Hg during progressive weaning from CPB and the use of inotropic or vasopressive support (norepinephrine >4 mg.min⁻¹, epinephrine >2 mg.min⁻¹, dobutamine >2 mg.kg⁻¹.min⁻¹) or the use of amrinone, milrinone, mechanical support or Intra Aortic Balloon Pump to be wean from bypass or to leave the operating room. The use of dopamine from 0.5-3.0 ug/kg/min is excluded in the definition.

The following compounds are also sometimes known under commercial names:
Prostacyclin (Flolan®)
Amrinone (Inocor®);
Nitroprussiate (Nipruss®);
Dobutamine (Dobutrex®); and
Milrinone (Primacor®).

Milrinone

Milrinone is drug that is currently used for reducing blood pressure. An inconvenient effect of this drug is that it likely reduces cardiac pressure, but it also reduces the systemic arterial pressure. Consequently some patients become more hemodynamically unstable further to the administration of milrinone.

More specifically, milrinone is a cyclic AMP specific phosphodiesterase inhibitor that can produce both positive inotropic effects and vasodilatation independently of β₁-adrenergic receptor stimulation in the cardiovascular system. This class of agents improves the response to β-adrenergic drugs and can potentiate the effects of dobutamine¹. Milrinone has in addition been demonstrated to improve diastolic performance in patients with congestive heart failure², left ventricular compliance after CardioPulmonary Bypass (CPB)^{2 3 4}, low cardiac output following CPB³⁻¹¹ and is superior to placebo in the CPB weaning process⁷.

Milrinone increases cardiac output and myocardial performance measured with transesophageal echography (TEE)^{12 3}. Its efficacy is comparable to amrinone¹³ and dobutamine¹¹. It also reduces systemic vascular resistance¹⁰ and pulmonary capillary wedge pressure¹¹. Randomized controlled trials on the use of milrinone in cardiac surgery are summarized in table 1.

A major difficulty with intravenous milrinone is the increased incidence of hypotension leading to an increase in the use of phenylephrine^{3 9} or norepinephrine¹⁰ to compensate for this hypotension. In addition, the use of intravenous milrinone is associated with an increased need for vasoactive support¹⁰ compared to nitric oxide (NO) therapy⁹, the latter being associated with a better improvement in right ventricular function.

A randomized trial on intravenous milrinone compared to placebo in 959 coronary care unit patients was recently published¹⁴. This trial shows that Milrinone, when compared to the placebo, leads to more sustained hypotension requiring intervention (10.7% vs 3.2%; P<0.001) and atrial arrhythmias (4.6% vs 1.5%; P=0.004). There was no difference in the median number of days hospitalized for cardiovascular causes within 60 days, in-hospital mortality (3.8% vs 2.3%; P=0.19) and 60-day mortality (10.3% vs 8.9%; P=0.41). These results do not support the routine use of intravenous milrinone as an adjunct to standard therapy in the treatment of patients hospitalized for an exacerbation of chronic heart failure. These results are not surprising as previous studies have demonstrated worse outcomes and increased mortality with inotropes^{15 16}.

As an alternative to intravenous milrinone, inhaled milrinone in patients with pulmonary hypertension has been demonstrated to reduce pulmonary vascular resistance and this effect was enhanced with the combined use of inhaled prostacyclin¹⁷.

Prostacyclin

Prostacyclin (PGI₂) is an endogen prostaglandin derived from arachidonic acid metabolism through the cyclooxygenase pathway synthesized mainly in the vascular endothelium. PGI₂ binds to a Gs-protein related receptor, which when activated, increases cyclic adenosine monophosphate (cAMP) concentration, activating a protein kinase A to decrease free intracellular calcium concentration. The physiological effects are vascular dilatation (predominantly in resistance vessels), inhibition of endothelin-1 secretion, inhibition of platelet aggregation and inhibition of leucocyte adhesion to the endothelium¹⁸.

More specifically, as shown in animal studies, intravenous PGI₂ has a short half-life of 2-3 minutes and is spontaneously hydrolysed at neutral pH in plasma to an inactive metabolite: 6-keto-PGI1a. Intravenous infusion of PGI₂ may increase intrapulmonary shunt and cause systemic vasodilatation that can be deleterious in hemodynamically unstable patients¹⁸.

Due to these systemic side effects, researchers have explored the bronchial tree as a route of administration, since the aerosolised form of PGI2 causes a selective dilatation of the pulmonary vessels and improves the right ventricular function and the cardiac output. Its effect remaining localized to ventilated lung units, it can decrease pulmonary artery pressure (PAP) without causing systemic hypotension and improve oxygenation by decreasing ventilation-perfusion mismatch¹⁹⁻²³.

Its effect on cardiac function when given by means of inhalation is controversial, but it can increase cardiac output when given intravenously^{23 24}. In one study comparing nitric oxide and inhaled prostacyclin in heart transplant candidate, the cardiac output increased by 11% in the prostacyclin group²⁵. The amount absorbed in the lung is controversial^{22 25} but the typical side effects seen with the intravenous administration (headache, jaw pain and facial flushing) are not seen with inhaled administration.

The effect on in-vivo platelet function has not been associated with an increase incidence of surgical bleeding^{26 27}. The effect of the addition of glycine buffer in a diluent has not been associated with pulmonary toxicity in an animal study during which the inhaled agent was administered for 8 hours²⁸.

In the cardiac surgery setting, PGI₂ has been used in clinical situations such as pulmonary hypertension and the adult respiratory distress syndrome (ARDS) and following CPB^{18 29 31 27}. Inhaled PGI₂ appears to be comparable with inhaled nitric oxide but acting through cyclic adenosine monophosphate instead of cyclic guanosine monophosphate^{25 21}. Its administration can be a simpler, less expensive alternative to inhaled nitric oxide and contrary to inhaled nitric oxide³² prostacyclin metabolites have no known toxic effects.

Clinical studies on inhaled prostacyclin are summarized in table 2. Experiences with inhaled prostacyclin in critical care patients and in the operating room³¹, during acute pulmonary hypertension from carbon dioxide embolism³³ in a randomized controlled trial on inhaled prostacyclin in patients with pulmonary hypertension undergoing cardiac surgery²⁷ have been reported. This last study demonstrated that inhaled prostacyclin in the pre-bypass period reduces pulmonary hypertension and that there was a tendency in the improvement of right ventricular diastolic dysfunction as measured by Doppler echocardiographic interrogation of the hepatic venous flow.

Notwithstanding the above, the impact of a prophylactic administration through inhalation of a vasodilatator on weaning from extra-corporal circulation and on mortality and morbidity following cardiac surgery involving extra-corporal circulation has not been previously assessed.

### SUMMARY OF THE INVENTION

Prophylactic strategies aimed at delivering vasodilators through inhalation in the pulmonary tree treat and prevent right ventricular dysfunction by reducing right ventricular afterload, facilitate separation from bypass and consequently decrease hemodynamic complications, morbidity and mortality.

In order to determine the impact of such a vasodilatator delivery, milrinone was administered to porcine subjects undergoing cardiac surgery involving extra-corporal circulation. The results of this study show that the prophylactic administration through inhalation of milrinone markedly reduces the stress caused by extra-corporal circulation on the organism.

This study strongly suggests that beneficial effects are obtainable from other compounds, such as prostacyclin, dobutamine, nitroglycerin, nitroprussiate and amrinone that are known to have effects similar to the effect of milrinone on the cardiovascular system in humans. Such beneficial effects on human subjects have been documented in a few subjects.

In a first broad aspect, the invention provides a method for reducing the severity of an hemodynamic instability in a subject undergoing a cardiac surgery involving an extra-corporal circulation. The method includes the administration through inhalation of a therapeutically effective amount of a vasodilatator to the subject.

Advantageously, the prognostic for the subject following the surgery is improved and the subject requires relatively little medication and other medical support to leave the operating room.

The administration is non-limitatively suitable when the hemodynamic instability is associated with a dilatation of the right ventricle. In some cases, this dilatation of the right ventricle is a result of a pulmonary hypertension in the subject and the vasodilatator dilates blood vessels within the lungs of the subject while substantially not dilatating blood vessels outside of the lungs of the subject.

In another broad aspect, the invention provides a method for reducing the morbidity of a subject in cardiac surgeries involving an extra-corporal circulation, the method including the administration through inhalation of a therapeutically effective amount of a vasodilatator to the subject.

In yet another broad aspect, the invention provides a method for facilitating weaning from extra-corporal circulation of a subject during a cardiac surgery, the method including the administration through inhalation of a therapeutically effective amount of a vasodilatator.

In yet other broad aspects, the invention provides the use of an inhaled vasodilatator for reducing the severity of an hemodynamic instability in a subject undergoing a cardiac surgery involving an extra-corporal circulation, the use of an inhaled vasodilatator for reducing the morbidity of a subject in cardiac surgeries involving an extra-corporal circulation, and the use of an inhaled vasodilatator for facilitating weaning from extra-corporal circulation of a subject during a cardiac surgery.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

Figure Legends

FIG. 1: A pathophysiological model of hemodynamic instability in cardiac surgical patients.

FIG. 2: Mean arterial pressure before (Pré Rx) and after (Post Rx) the administration of intravenous (IV) and inhaled milrinone in porcine subjects.

FIG. 3: Systemic vascular resistance before (Pré Rx) and after (Post Rx) the administration of intravenous (IV) and inhaled milrinone in porcine subjects.

FIG. 4: Mean arterial pressure as a function of time for porcine subjects undergoing cardiopulmonary bypass (CPB) further to the administration of intravenous (IV) and inhaled milrinone and for subjects undergoing CPB without administration of milrinone (CPB curve). Data is presented before (Pré Rx and Post Rx), during (per) and after (post) extra-corporal circulation.

FIG. 5: Cardiac index as a function of time for porcine subjects undergoing cardiopulmonary bypass (CPB) further to the administration of intravenous (IV) and inhaled milrinone and for subjects undergoing CPB without administration of milrinone (CPB curve). Data is presented before (Pre Rx and Post Rx), during (per) and after (post) extra-corporal circulation.

FIG. 6: Heart rate as a function of time for porcine subjects undergoing cardiopulmonary bypass (CPB) further to the administration of intravenous (IV) and inhaled milrinone and for subjects undergoing CPB without administration of milrinone (CPB curve). Data is presented before (Pré Rx and Post Rx), during (per) and after (post) extra-corporal circulation.

FIG. 7: Alveolo-arterial oxygen gradient as a function of time for porcine subjects undergoing cardiopulmonary bypass (CPB) further to the administration of intravenous (IV) and inhaled milrinone and for subjects undergoing CPB without administration of milrinone (CPB curve). Data is presented before (Pré Rx and Post Rx), during (per) and after (post) extra-corporal circulation. Deterioration of the alveolo-arterial oxygen gradient is seen with intravenous milrinone but not inhaled milrinone.

FIG. 8: Mean pulmonary artery pressure as a function of time for porcine subjects undergoing cardiopulmonary bypass (CPB) further to the administration of intravenous (IV) and inhaled milrinone and for subjects undergoing CPB without administration of milrinone (CPB curve). Data is presented before (Pré Rx and Post Rx) and after (post) extra-corporal circulation

FIG. 9: Tension in rings of porcine pulmonary artery with endothelium as a function of the concentration of ACh for samples taken in subjects further to no extra-corporal circulation (control), extra-corporal circulation without the administration of milrinone (CPB curve) and extra-corporal circulation with the administration of intravenous (IV) and inhaled milrinone. The control group (without extra-corporal circulation) behaves similarly to the inhaled milrinone group, indicating preventive effect of inhaled milrinone on endothelial function. This effect is not seen with intravenous milrinone.

FIG. 10: Tension in rings of porcine pulmonary artery with endothelium as a function of the concentration bradykinin (BK) for samples taken in subjects further to no Extra-corporal circulation (control), extra-corporal circulation without the administration of milrinone (CPB curve) and extra-corporal circulation with the administration of intravenous (IV) and inhaled milrinone. The control group (without extra-corporal circulation) behaves similarly to the inhaled milrinone group, indicating preventive effect of inhaled milrinone on endothelial function. This effect is not seen with intravenous milrinone.

### DESCRIPTION OF THE PREFERRED EMBODIMENTS

Introductory Remarks

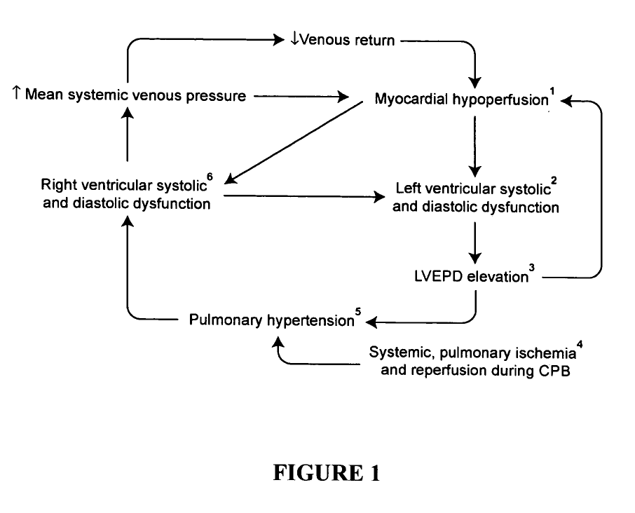

From available animal and human clinical data, the following pathophysiological model of hemodynamic instability in cardiac surgical patients, illustrated in FIG. 1, is produced.

Myocardial hypoperfusion leads and predisposes to systolic and diastolic dysfunction. With progression of the phenomenon, elevation in Left Ventricular End Diastolic Pressure (LVEDP) occurs, which in turn may lead to secondary pulmonary hypertension and right ventricular systolic and diastolic dysfunction. Pulmonary hypertension is also be exacerbated with the pulmonary ischemia reperfusion injury after CPB and the inflammatory response to the CPB circuit and the effect of pre-operative or intraoperative tissue hypoperfusion.

In addition, through interventricular interdependence, pulmonary hypertension exacerbates left ventricular diastolic dysfunction leading to more pulmonary hypertension. The final result is a progressive reduction in venous return and cardiac output though increased right sided pressures and signs of right sided failure with associated hemodynamic instability.

Therefore, from the above and from published studies, the following hypotheses on hemodynamic instability after cardiac surgery are formulated:

1—Increased veno-arterial Carbon Dioxyde partial pressure (PCO₂) before (CPB) is an independent factor for difficult separation from bypass (DSB)⁵².

2—Left ventricular diastolic dysfunction⁵³ and right ventricular diastolic dysfunction predisposes to hemodynamic instability and DSB.

3—Elevated (LVEDP) predisposes to hemodynamic instability, DSB and death⁵⁴.

4—Pulmonary ischemia and reperfusion during CPB is associated with pulmonary hypertension and prevented by inhaled prostacyclin⁵⁵ and global ischemia during CPB increases hemodynamic instability and death⁵².

5—Pulmonary hypertension predisposes to hemodynamic instability⁵⁸. Inhaled prostacyclin reduces pulmonary hypertension and the incidence of hemodynamic instability^{56 57}.

6—Right ventricular systolic and diastolic dysfunction is commonly present in hemodynamic instability⁵⁹.

Myocardial hypoperfusion chronically or acutely, before and after CPB either through coronary artery disease, poor myocardial protection, clots, air or carbon dioxide embolism during the cardiac procedure and poor cardiac output could lead and predispose to systolic and diastolic dysfunction. As the disease progresses, gradual elevation in LVEDP and secondary pulmonary hypertension⁶⁰ may ensue. Pulmonary hypertension may be exacerbated by ischemia reperfusion after CPB and pre-operative or intraoperative global and regional hypoperfusion.

Pulmonary hypertension will eventually lead to progressive right atrial^{61 62} and ventricular dilatation which is associated with abnormal right ventricular systolic and diastolic function. In addition, through ventricular interdependence and ventricular septal shift, pulmonary hypertension could exacerbate left ventricular diastolic dysfunction⁶³ leading to more severe pulmonary hypertension. The final result is a progressive reduction in venous return and cardiac output through increased right sided pressures and signs of right sided failure with associated hemodynamic instability.

In view of the above, it can be hypothesized that the prophylactic administration through inhalation of a suitable vasodilatator would reduce hemodynamic instability during and after surgeries involving extra-corporal circulation, which would in turn reduce morbidity and mortality in this type of intervention.

Animal Study

An animal study was performed to asses the effects of inhaled and intravenous milrinone on the alterations of pulmonary endothelium-dependent relaxations, hemodynamic and oxygenation parameters after CPB in a porcine model.

In summary, four groups of Landrace swine were compared: 1—control group: without CPB; 2—CPB group: 90 minutes of normothermic CPB and 60 minutes reperfusion; 3—Inhaled milrinone: 90 minutes of CPB and 60 minutes reperfusion, preceded by inhaled milrinone; 4—Intravenous milrinone group: 90 minutes of CPB and 60 minutes reperfusion. After 60 minutes of reperfusion, swine were sacrificed and pulmonary arteries harvested. After contraction to phenylephrine, pulmonary arteries endothelium-dependent relaxations to bradykinin (Gq coupled) and acetylcholine (Gi coupled) were studied in standard organ chamber experiments.

Inhaled milrinone caused less hypotension and lowering of the peripheral vascular resistances than intravenous milrinone. The heart rate was significantly lower in the inhaled milrinone group than in the CPB and the intravenous milrinone group. Intravenous milrinone caused a significant increase in the alveolo-arterial oxygen gradient. CPB caused a statistically significant decrease in endothelium-dependent relaxations to acetylcholine (ACh). There was a significant improvement of the endothelium-dependent relaxation to ACh and to bradykinin in the inhaled milrinone group (p<0.05). Intravenous milrinone did not reverse pulmonary endothelial dysfunction. Endothelium-independent relaxations to sodium nitroprussiate were unaltered.

In conclusion, prophylactic use of inhaled milrinone reverses pulmonary endothelial dysfunction following CPB. The hemodynamic and oxygenation profile of inhaled milrinone is safer than intravenous milrinone. These strategies may be usefull in prophylaxis of post CPB pulmonary hypertension after cardiac surgery.

More details regarding this study are given hereinbelow.

Introduction

Cardiopulmonary bypass (CPB) induces a systemic inflammatory response that alters a majority of the organ systems. The physiological alterations following CPB where recognized early after the development of CPB in the 1950s. The post pump syndrome is characterised by an increase in pulmonary capillary permeability leading to a decreased oxygenation and an increased AaDO2. The pulmonary compliance is decreased, and the pulmonary vascular resistance is increased. Some of the most important repercussions of that inflammatory cascade are on the pulmonary vasculature. During CPB, the blood flow is diverted from the right atrium to the CPB pump, flows trough an oxygenator membrane and pumped back into the aorta. Thus, the lungs are not perfused. At the separation from CPB, lungs are reperfused and suffer from ischemia-reperfusion injury, with an exposition to important amounts of free radicals. The blood being in contact with the non physiological surface, neutrophils and platelets are activated and contribute to pulmonary damage. Several authors have reported endothelial dysfunction following CPB.

The endothelium has an important role as a regulator of the vascular tone, of platelet aggregation and of neutrophil adhesion. It liberates several vasoactive substances which can be classified in Endothelium Derived Relaxing Factors (EDRF), as nitric oxide (NO) and prostacyclin, and Endothelium Derived Contracting Factors (EDCF) as endothelin (ET-1) and oxygen free radicals. When the endothelial integrity is altered, synthesis of relaxing factors is decreased. Endothelial dysfunction can be defined as an imbalance between relaxing factors and contracting factors, and results in the loss of the normal protective role of the endothelium in the homeostasis of the vascular wall.

After CPB, the endothelial damage to the pulmonary endothelium can lead to pulmonary hypertension. This pulmonary hypertension increases the right ventricular work. Right ventricular dysfunction following CPB carries a very bad prognosis with a perioperative mortality ranging from 44% to 86%. Several pharmacologic agents have been used to try to limit the pulmonary hypertension following cardiac surgery including intravenous nitroglycerin, intravenous milrinone, inhaled NO and inhaled prostacyclin

Prostacyclin (PGI2) is an endogenous prostaglandin derived from arachidonic acid metabolism through the cyclooxygenase pathway in the vascular endothelium. PGI2 binds to a Gs-protein related receptor, which, when activated, increases cyclic adenosine monophosphate (cAMP) concentration, activating a protein kinase A to decrease free intracellular calcium concentration. The physiological effects are vascular dilatation (predominantly in resistance vessels), inhibition of endothelin secretion, inhibition of platelet aggregation and inhibition of leukocyte adhesion to the endothelium. Prostacyclin secretion is one of the factors that can act as a vasodilator in the event of reduced NO biodisponibility. During CPB, circulating levels of PGI2 are supranormal and decrease following separation from CPB. These decreased levels in the prostacyclin venous concentration following CPB are accompanied by an increase in pulmonary artery pressure. It is demonstrated that CPB damages pulmonary endothelial function, limiting NO secretion, also contributing to pulmonary hypertension.

Milrinone is a phosphodiesterase III inhibitor. Phosphodiesterase III metabolises cAMP, thus milrinone increases the intracellular levels of cAMP. Systemic effects of milrinone are cardiac positive inotropy and diffuse vasorelaxation by acting on membrane calcium permeability. Milrinone is used in cardiac surgery patients to treat low cardiac output and pulmonary hypertension. When given intravenously, milrinone decreases the systemic vascular resistances, which can be hazardous in the hours following cardiac surgery, while vasopressor drugs are frequently used. The use of inhaled milrinone has recently been described by⁶⁴. The use of inhaled milrinone prior to surgery in cardiac surgical patients with pulmonary hypertension lowered pulmonary vascular resistances without any systemic hypotension. The aim of this study is to compare the effects of inhaled and intravenous milrinone in a swine model of cardiopulmonary bypass on pulmonary endothelial function, hemodynamics and oxygenation. The levels of cyclic AMP and GMP will also be compared to document the mechanism of action of the drug.

Material and Methods

Experimental Preparation for all Groups (Anaesthesia)

All experiments were performed using Landrace white swine (McGill University, Montreal, QC) of either gender, aged 8 weeks and weighing 25+/−2.9 kg. Animals were maintained and tested in accordance with the recommendations of the guidelines on the Care and Use of Laboratory Animals issued by the Canadian Council on Animal and were approved by a local ethics committee. The piglets were fasted for 12 hours prior to surgery and were sedated with intramuscular ketamine hydrocloride (25 mg/kg)(Ayerst Veterinary Laboratories, Guelf, ON) and Xylazine (10 mg/kg)(Boehringer Ingelheim, Burlington, ON) and induction was achieved using mask ventilation with 2% isoflurane (Abbott Laboratories Limited, St-Laurent, QC). They were subsequently intubated and mechanically ventilated with oxygen and air mixture (3:2, or FiO2=0.66) at 14 breaths/min and tidal volume of 6-8 ml/kg. Anaesthesia was maintained with 1% isoflurane inhalation. Arterial and venous blood gases were measured at regular intervals and maintained within physiological limits by adjusting the inspired oxygen fraction (FiO2), ventilation rate and tidal volume. The electrocardiogram was recorded from four subcutaneous limb and one precordial electrode.

Experimental Groups

Group 1: Control

After skin preparation, the mediastinum was exposed via median sternotomy. 300 UI/kg heparin (Leo Pharma Inc. Ajax, ON) were given intravenously. After 1 hour of general anaesthesia with 1% isoflurane, the animal was exanguinated and the lungs harvested.

Group 2: Cardiopulmonary Bypass

After skin preparation and draping with sterile fields, the jugular vein and the carotid artery were cannulated to obtain a central venous line and arterial pressure, respectively. A cystostomy was performed for urine output measurement. A median sternotomy was performed and the pericardium opened for heart exposition. A Swan-Ganz catheter (Edwards Lifesciences, Irving, Calif.) was inserted through the jugular vein to measure pulmonary artery pressure. After heparin administration (400 UI/kg), a double purse string was made on the proximal ascending aorta and a single purse string on the right atrium. A blood sample was drawn thereafter from the right atrium and proper anticoagulation assessed using an activated coagulation time (ACT) with hemochron 801 (Technidyne, N.J., USA). The aorta and right atrium were cannulated when ACT was superior to 300 seconds, with a 22-Fr and a 29/29 Fr double staged cannulas (DLP, Inc., Grand Rapids, Mich., USA), respectively. After cannulation, CPB was initiated when ACT was superior to 400 seconds. Ventilation was stopped throughout the CPB period. Anaesthesia was maintained using the jugular vein line with a continuous infusion of propofol (0.1-0.2 mg/kg/min). The CPB circuit consisted of a hollow fiber membrane oxygenator with incorporated filtered hardshell venous reservoir (Monolyth, Sorin, Irvine, Calif., USA), a heater-cooler and a roller pump (Sarns 7000, Ann Harbor, Mich., USA). The circuit was primed with Pentaspan 500 mL (10% Pentastarch, DuPont Pharma Inc, Mississauga, ON, Canada), lactated Ringer's 250 mL, heparin 5000 UI, mannitol 12.5 g and sodium bicarbonate 15 mEq. After initial stabilization, the pump flow was adjusted to obtain an index of 2.4 L/min/m² and assessed by venous gases to maintain mixed venous saturation over 60%. Mean systemic arterial pressure was maintained between 50 and 70 mm Hg with crystalloid (Ringer's lactate) and punctual boluses of 50 to 200 μg of neosynephrine (Cayman Chemical Company, Ann Arbor, Mich., USA). The temperature was allowed to drift to 36° C. The heart was left beating, empty. No aortic cross clamping or cardioplegia was used. Before CPB weaning, swine were rewarmed to 38° C. (normal porcine temperature). After 90 minutes of CPB, mechanical ventilation and isoflurane anaesthesia were reinstituted and CPB was weaned. Normal circulation was restored for 60 minutes, at which time the animal was exsanguinated into the cardiotomy reservoir. The beating heart and the lungs were excised “en bloc” and immediately immersed in a cold modified Krebs bicarbonate solution (composition in mmol/L: NaCl 118.3, KCl 4.7, MgSO4 1.2, KH2PO4 1.2, glucose 11.1, CaCl2 2.5, NaHCO3 25, and ethylenediaminotetraacetic acid 0.026).

Group 3: Cardiopulmonary Bypass and Inhaled Milrinone (n=6)

The same procedure was followed as in the CPB group (group 2). The only difference was that a bolus of 60-90 mg/kg of milrinone (Primacor, Sanofi) was given via the endotracheal tube through a nebulizer during the 15 minutes preceding the initiation of CPB. Ventilation was then stopped but a continuous nebulisation of milrinone at a rate of 7-10 μg/kg/min with CPAP of 3 cm H2O was then instituted until the end of the CPB. Before weaning of CPB, nebulisation was stopped and ventilation reinstituted. Milrinone was given as a dilution of 2 mg of milrinone 1 mg/ml diluted in 8 ml of normal saline (200 μg/ml). The drug was administered through a conventional in-line nebulizer kit (Salter Labs, Arvin, USA) connected to the inspiratory limb of the ventilator.

Group 4: Cardiopulmonary Bypass with Intravenous Milrinone.

The same procedure was followed as in the CPB group (group 2). The only difference was that 2 mg of milrinone was diluted in 10 ml of saline solution and administered intravenously over 15 minutes after the administration of heparin.

Hemodynamic and Biochemical Data

Heart rate was continuously recorded from 5 subcutaneous limb electrodes. Arterial and venous blood gases were measured at regular intervals during the experiment (baseline, during CPB at 15, 45 and 75 minutes, and at 30 and 60 minutes after weaning from CPB) and maintained within physiological limits by adjusting ventilation rate and tidal volume. Hemodynamic parameters such as mean arterial pressure, heart rate, mean pulmonary artery pressure, central venous pressure and pulmonary artery wedge pressure were measured with a Swan-Ganz catheter at different intervals of the procedure: after induction, after drug administration and after weaning of CPB (30 minutes and 60 minutes).

Vascular Reactivity Studies

Less than 10 minutes after “en bloc” excision, the heart was removed and the primary pulmonary artery was dissected. Branches of second degree pulmonary arteries were isolated and dissected free of connective and adventitial tissue and divided into rings (4 mm wide; 16 rings per animal). All rings were placed in organ chambers (Emka technologies Inc, Paris, France) filled with 20 mL modified Krebs-bicarbonate solution continuously heated at 37° C. and oxygenated with a carbogen mixture (95% O₂ and 5% CO2). The rings were suspended between 2 metal stirrups with the upper 1 connected to an isometric force transducer connected to a signal amplifier and then allowed to stabilize for 30 minutes. Data were collected with a biological signal data acquisition software (IOX 1.700; Emka technologies Inc, Paris, France). Each arterial ring was stretched to the optimal point of its active length-tension curve (3.5 g) as determined by measuring the contraction to potassium chloride (KCl) 60 mmol/L at different levels of stretch (data not shown). The maximal contraction of rings was then obtained with addition of potassium chloride (KCl 60 mmol/L). After obtention of a plateau, all baths were washed twice with modified Krebs bicarbonate solution and indomethacin (10-5 mmol/L; to exclude production of endogenous prostanoids) was added in each bath. After 45 minutes of stabilisation, phenylephrine (range 2 ´ 10-7 mol/L to 3 ´ 10-6 mol/L) was added to obtain a contraction averaging 50% of the maximal contraction to KCl.

Endothelium-Dependent Relaxations

The NO-mediated relaxation pathway was studied by constructing concentration-response curves to acetylcholine (ACh, 10-9 to 10-5 mol/L; an agonist of M2 receptors coupled to Gi-proteins) and to bradykinin (BK, 10-12 to 10_{—}6 mol/L; an agonist of B2 receptors coupled to Gq-proteins).

Endothelium-Independent Relaxations

At the end of the experiment, endothelium-independent relaxations were studied with the use of 10-5 mol/L sodium nitroprusside (SNP), a nitric oxide donor

Study Drugs

All drugs were prepared daily. Acetylcholine, bradykinin, indomethacin, and sodium SNP were obtained from Sigma Chemical Co. (ON, Canada). Propranolol was obtained from Biomol Research Laboratories, Inc. (Plymouth Metting, Pa., USA) and phenylephrine was obtained from Cayman Chemical Company (Ann Arbor, Mich., USA). Milrinone was obtained from Sanofi Synthelabo (Markham, ON, Canada)

Determination of Pulmonary Artery Intravascular Cyclic AMP and Cyclic GMP Content.

To determine the vascular cyclic AMP content of porcine pulmonary arteries, rings from the 3 groups were collected after sacrifice, frozen in liquid nitrogen and stored at −70° C. At the time of analysis, all segments were pulverized in a liquid nitrogen-cooled stainless steel mortar, and then transferred in trichloracetic acid solution (TCA; 6.25% w/v). The acid extracts were then centrifuged at 4° C. for 15 minutes at 12,000 g (3000 RPM) to precipitate cell debris and proteins. The pellets were used for total protein determination using the Bradford microassay technique (Bio-Rad, Mississauga, ON, Canada). To remove trichloracetic acid, the supernatants were extracted 4 times with water-saturated diethyl ether. Any residual diethyl ether was removed by heating the samples to 90° C. for 3_{—}5 minutes. Cyclic AMP and cGMP quantitation was done using an enzyme immunoassay (EIA) system with acetylation based on rabbit anti-cAMP and anti-cGMP antibodies (Amersham Pharmacia Biotech, Baie d'Urfé, QC, Canada). The amount of cyclic AMP and cGMP in each blood vessel ring was standardized to pmol cyclic AMP·mg-1 protein and pmol cyclic GMP·mg-1 protein.

Statistical Analysis

All values are expressed as the mean±standard error of the mean (SEM). Contractions to phenylephrine are expressed as a percentage of the maximal contraction to KCl (60 mmol/L). Relaxations are expressed as the percentage of the maximal contraction to phenylephrine for each ring. Two-way repeated analysis of variance (ANOVA) were performed to compare each point of the concentration-response curves between control rings and CPB rings. Student's t test for paired/unpaired observations was used for the comparison of the pulmonary artery pressures and the intravascular cAMP content. Statistical analysis was performed with the computer software S.A.S (Instr Inc. Cary, N.C., USA). A P-value less than 0.05 was considered statistically significant.

Results

Hemodynamic and Biochemical Data

Effect of the Milrinone Bolus (Inhaled or Intravenous)

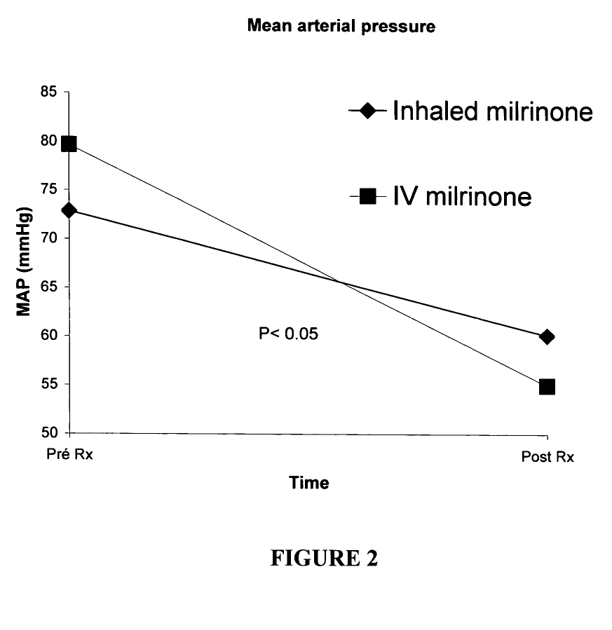

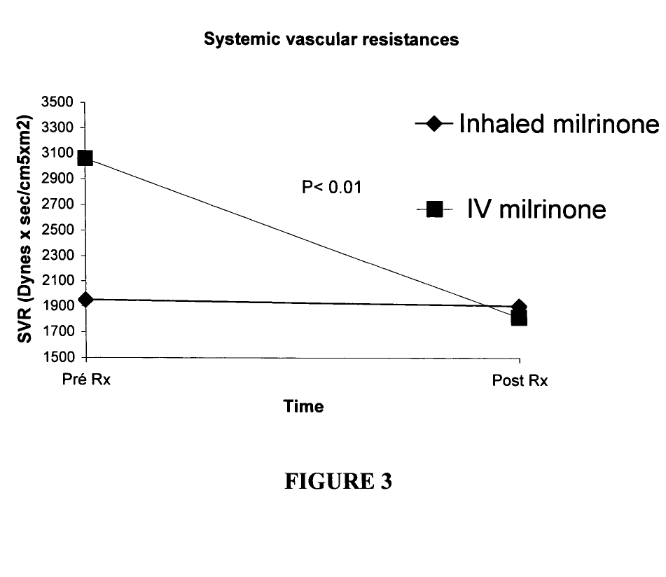

There was a significant decrease in the mean arterial pressure following administration of the inhaled and intravenous milrinone bolus (p<0.05). The decrease was significantly more important in the intravenous milrinone group (p<0.05) (FIG. 2). We observed an important decrease in systemic vascular resitance in the intravenous milrinone group while the resistance did not fall in the inhaled milrinone group. (p<0.01) (FIG. 3)

Hemodynamic Data During and After Bypass

Blood Pressure and Cardiac Index

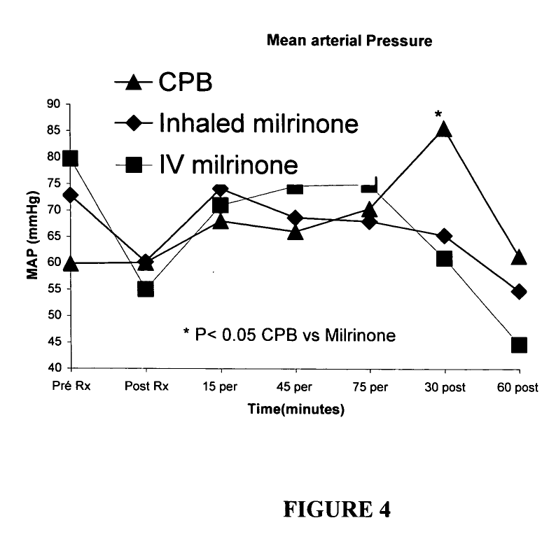

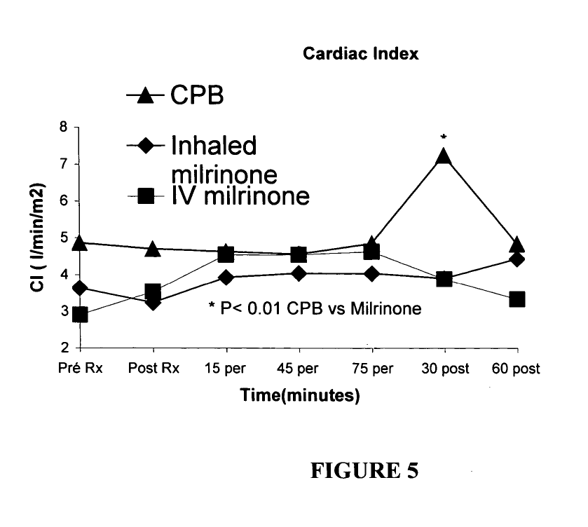

In the CPB group, the mean arterial pressure and cardiac index were stable throughout the study period except for the time 30 minutes post weaning of CPB, where there was a significant increase in the blood pressure. (p<0.05) (FIG. 4) and cardiac index (p<0.01) (FIG. 5). Inhaled and intravenous milrinone blunted this peak 30 minutes after the end of bypass.

Heart Rate

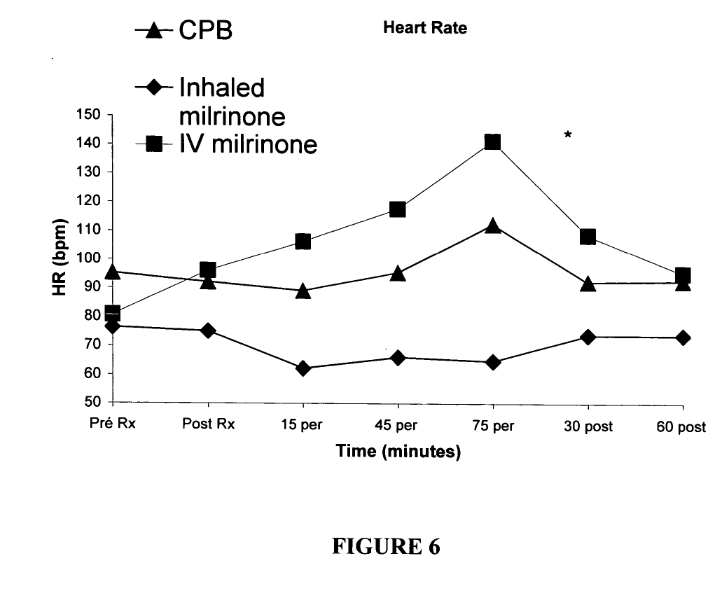

There was a slight increase in the heart rate during the CPB time in the CPB group (p=NS) (FIG. 6). The heart rate was increased compared to the CPB group in the intravenous milrinone group. The high heart rate reached statistical significance only at 75 minutes of bypass (p<0.05). Heart rate was significantly lower in the inhaled milrinone group (vs CPB) at 15, 45 and 75 minutes per CPB and at 30 and 60 minutes post CPB. (p<0.05)

Oxygen Exchanges

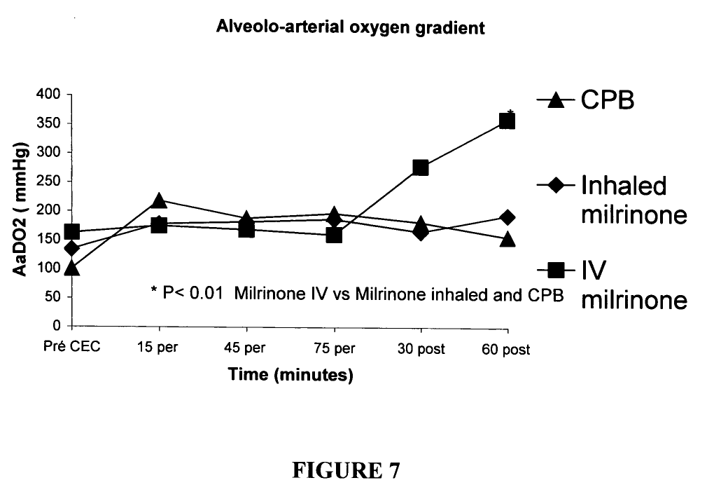

The alveolo-arterial oxygen gradient was significantly greater in the intravenous milrinone group at 60 minutes after bypass (p<0.05) (FIG. 7) comparing to CPB and inhaled milrinone group. Oxygen exchange was not different in the inhaled milrinone and in the CPB group.

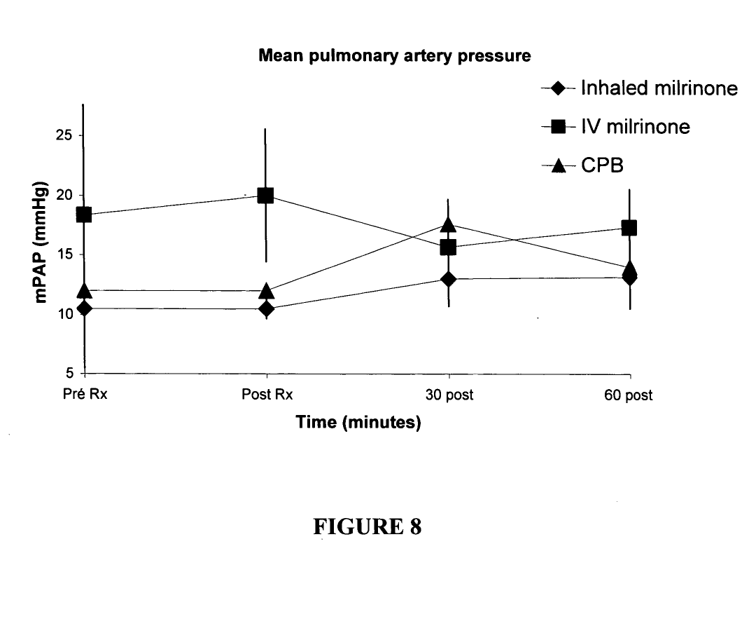

There was no statistical difference in the pulmonary artery pressure during the experiment inside or between the groups. (FIG. 8)

Vascular Reactivity Studies

Contractions

The amplitude of the contraction to KCl (60 mmol/L) and the concentration of PE used to obtain 50% of contraction to KCl were quantified for both groups in Table 3. The amplitude of contraction to KCl (endothelium-independent agent) was not significantly different between the groups. The dose of phenylephrine necessary to obtain 50% of the contraction to KCl was greater in the inhaled milrinone group vs control (p<0.05) and vs intravenous milrinone (p<0.01).

Relaxation

Endothelium-Dependent Relaxation

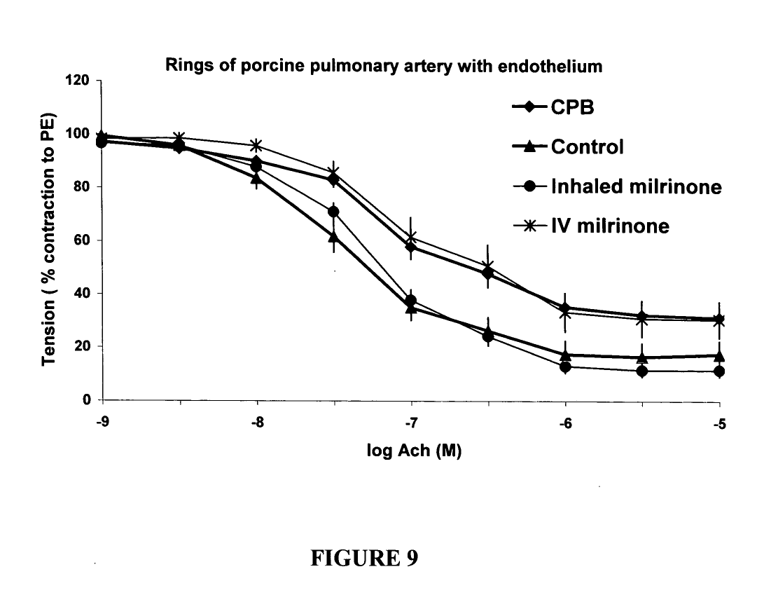

There was a statistically significant decrease of endothelium-dependent relaxation to ACh in the CPB group when compared to controls (P<0.05) (FIG. 9). This decrease in relaxation was completely reversed by the administration of inhaled milrinone prior to CPB, but not by intravenous milrinone, which was not different than CPB alone.

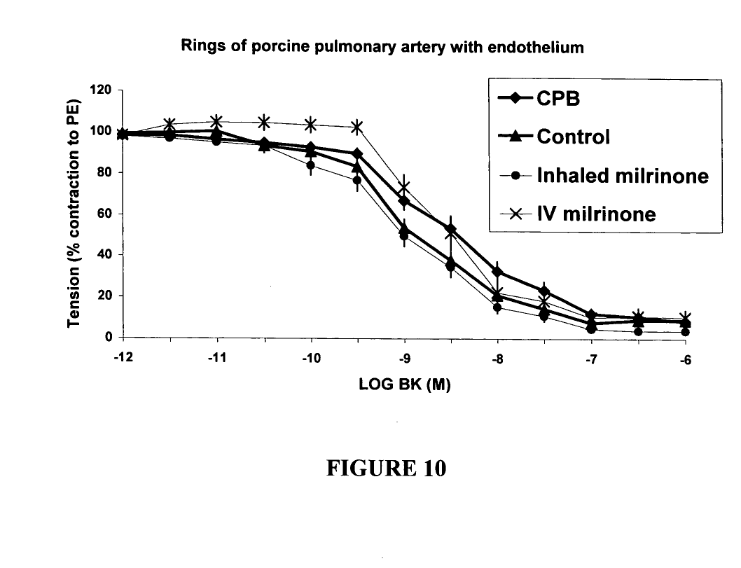

There was a no statistically significant difference in endothelium-dependent relaxation to BK in the CPB when compared to the control group (P<0.05). There was an increased relaxation in the inhaled milrinone group compared to all other groups (P<0.05) (FIG. 10).

Endothelium-Independent Relaxation

No statistically significant difference in relaxation to the SNP was observed between groups with all rings achieving 100% relaxation.

Discussion

The aim of this study was to compare the effects of inhaled and intravenous milrinone boluses before cardiopulmonary bypass. The major findings of this study are that 1) The dose of phenylephrine used to contract pulmonary arteries were higher in the inhaled milrinone group. 2) CPB induces pulmonary endothelial dysfunction selective to the ACh pathway. 3) This dysfunction is reversed by administration of inhaled milrinone prior to CPB. 4) Relaxation following stimulation by BK is enhanced in swine exposed to inhaled milrinone. 5) Inhaled milrinone is associated with a better hemodynamic profile than intravenous milrinone, with less hypotension after administration and a lesser drop in systemic vascular resistances. 6) During CPB, Inhaled milrinone is associated with a decrease in the heart rate compared to IV milrinone and CPB alone. 7) Intravenous milrinone is associated with an increased alveolo-arterial oxygen gradient.

Several types of cardiac surgery can be followed by pulmonary hypertension (PH). Mitral valve surgery and coronary artery bypass grafting (CABG) with left ventricular dysfunction frequently present to the hospital with preexisting PH and are prone to develops this pathology in the postoperative setting. Pulmonary hypertension increases right ventricular work, which can lead to right ventricular dysfunction. This pathology carries a poor prognosis. Morita and colleagues 65 demonstrated in a porcine model that CPB causes a significant increase in pulmonary vascular resistance and depresses the RV function by more than 50%. Pulmonary artery endothelial dysfunction is characterized by an decrease in the secretion of relaxing factors. After separation from CPB, the imbalance toward contracting factors result in pulmonary hypertension, leading to RV dysfunction and low cardiac output syndrome.

We compared two modes of administration of a frequently used drug in the post CPB setting. Only one study mentions the use of inhaled milrinone after cardiac surgery. As previously described, our model of CPB in swine is reproducible and is associated with postoperative pulmonary endothelial dysfunction.

In this study, the doses of phenyephrine used to contract pulmonary arteries were higher in the inhaled milrinone group than in the control and in the intravenous milrinone group. These higher doses may reflect a relative pulmonary vasoplegia potentially caused by increases bioavailability of cAMP. A low response of vascular smooth muscle cells (SMC) to contracting agents could have some beneficial implication in a state of lower endothelial NO production. The hemodynamic effect of this vasoplegia could be a lower pulmonary vascular resistance in the inhaled milrinone group.

The lower relaxations to ACh in the CPB group compared to controls were completely reversed by inhaled milrinone but not by intravenous milrinone. The relaxations to BK were greater in the inhaled milrinone group than in the three other groups. Milrinone acts as an inhibitor of type III phosphodiesterase. Thus, it increases the levels of cAMP in the smooth muscle cells. cAMP creates a vasorelaxation by lowering intracellular calcium levels, inhibiting muscle contraction. We tested the relaxations to ACh and to BK in the absence of PGI2 production by the endothelium, the rings being treated with indomethacin. Consequently, the increased relaxation must be due to increased NO production or to increased response to NO in the SMC. Fortier et al⁵⁵ showed recently that an inhaled PGI2 loading prior to CPB was associated with increased endothelium dependent relaxations to BK. This also favor the hypothesis that increased cAMP enhances the secretion of NO by the endothelial cell or sensitizes the smooth muscle cell to its effect. Niwano et al⁶⁶ described in 2003 the presence of a cAMP responsive element (CRE) in the endothelial cell DNA that enhances the synthesis of eNOS. His team reported the use of beraprost, a PGI2 receptor agonist, to enhance cAMP levels. The high cAMP levels where associated with higher eNOS expression and enhanced NO bioavailability. Milrinone may then increase levels of cAMP, promoting the secretion of NO by the endothelial cells.

The reason why the same drug achieved different effects on the endothelial-smooth muscle cell complex is not clear. Intravenous milrinone has an important distribution volume, the amount of this drug that reach the lungs is unknown. Furthermore, the adverse hemodynamic effects, as tachycardia and hypotension, may adversely affect the pulmonary endothelial function. Inhaled milrinone was administered as a bolus before initiation of CPB, followed by a continuous nebulisation throughout CPB time. The amount of milrinone reaching the lungs by nebulisation was not studied in the present experiment. The administration of milrinone by nebulisation should not induce V/Q mismatch since only vessels of ventilated regions of the lung are reached by the molecule. Intravenous milrinone dilated the vessels in a more general fashion, explaining the higher AaD_{O2} in that group. We can expect that the role of the initial bolus of inhaled milrinone carried a much more important effect than the continuous nebulisation because during CPB, only a 3 cm H₂O PEEP was applied to the lungs, without ventilation, not favoring deposition of particules far in the parenchyma. The effect of CPAP during CPB was studied by Lockinger et al.⁶⁷ Their team reported that a 10 cm H₂O CPAP was associated with better V/Q match after CPB. Our PEEP was lower and we did not observe any change in the AaDO2 following CPB in the inhaled milrinone group.

We did not observe a significant difference in the pulmonary artery pressure or the pulmonary vascular resistance in the different groups. This may be due to the important variations seen between the animals. The wide variation of the hemodynamic parameters and the small number of animals are probably responsible for that lack of statistical difference.

The lower drop in arterial pressure and in the systemic vascular resistance in the inhaled milrinone group compared to its intravenous counterpart is interesting. Intravenous milrinone is well known for its systemic vasodilating effect. The inhaled route seems to be associated with a safer profile, with lower systemic actions. We also observed that the tachycardia associated with the CPB was reversed by the inhaled milrinone, with a relative bradycardia. The intravenous milrinone increased the heart rate compared to CPB. The decrease in myocardial oxygen demand associated with a slower heart rate is an advantage for the inhaled milrinone. The lower heart rate with a stable cardiac output means that the ejection volume is increased in the inhaled milrinone group, this is another advantage for the inhaled milrinone.

Clinical Relevance

Cardiopulmonary bypass is used everyday in cardiac surgery and despite a relatively low prevalence of postoperative pulmonary hypertension, a certain level of pulmonary endothelial dysfunction is present in most of the patients with or without clinically apparent manifestations. On the other hand, risk factors for postoperative pulmonary hypertension are well known and the patients could benefit from prophylactic agents to lower their risk of developing this pathology and its consequence. We present a new mode of administration for a well studied drug that has been used for years in cardiac surgery. That drug is associated with less hypotension than the IV form and positively affect the oxygen exchange comparing to the IV administration. It was not associated with adverse events in any of our swine. We now have a locally acting drug that can reverse endothelial dysfunction in the lung, the only organ exposed to ischemia reperfusion during CPB.

Conclusion

A study comparing the effects of two modes of administration of a commonly used drug in swine undergoing CPB was conducted. The administration of inhaled milrinone was safer and was associated with a lower heart rate throughout surgery. It completely reversed endothelial dysfunction and was associated with better oxygen exchanges than its intravenous counterpart in this pulmonary ischemia-reperfusion model. These results suggest that therapy might be useful in patients at risk for postoperative pulmonary hypertension undergoing cardiac surgery and in other examples of ischemia-reperfusion injuries like lung transplant.

Extension to Human Subjects

The above-described animal study strongly suggests that beneficial effects are obtainable from the inhalation of milrinone prior to extra-corporal circulation in mammals other than pork, for example in humans. In addition, other compounds, such as prostacyclin, dobutamine and amrinone are known to have effects similar to the effect of milrinone on the cardiovascular system in humans. Notably, all these compounds are vasodilatator.

Some data acquired on human subjects also show the beneficial effects of milrinone and prostacyclin. For example, 5 mg of milrinone was administered through inhalation to a woman in cardiogenic shock. Echocardiographic monitoring of this subject showed that the administration was associated with a reduction in the E/A ratio of the trans-mitral flux, an increase in the S/D ratio of the pulmonary venous flux and a significant decrease in the Em/Am ratio for the mitral annulus obtained from Doppler imaging. Hemodynamic monitoring of this subject showed that the administration was associated with an increase in heart rate from 72 to 84 beats/minutes, a decrease in mean arterial pressure from 92 to 77 mm Hg, a decrease in mean pulmonary arterial pressure from 33 to 24 mm Hg and a decrease in right atrial pressure (RAP) from 17 to 7 mm Hg. Finally, the cardiac index increased from 1.8 to 2.8 L.min/m\hat{ }2. This strongly suggest that inhaled milrinone improves both systolic and diastolic function.

In another case, a 23 years-old man was operated for a third time under cardiopulmonary bypass. He had an endocarditis of the mitral prosthesis. Pre-operatively, he had abnormal right ventricular diastolic function with a lower systolic or S wave on the hepatic venous Doppler signal. After bypass (duration 157 min), he was weaned with inhaled prostacyclin (75 μg) and he left the operating room with noradrenaline at 2.5 μg/min and nitroglycerine at 0.4 μg/kg/min. The hepatic venous flow signal did not change significantly and the right ventricular diastolic waveform was still abnormal after the procedure. No vasoactive drugs were required in the post-operative period. The hemodynamic profile after bypass showed an increase in the cardiac index from 2.3 to 3.3 L/min/m² with a reduction in the pulmonary vascular resistance indexed from 242 to 121 dynes.s/cm⁵/m². Heart rate and mean arterial pressure respectively increased and decreased from 66 to 85 beats/min and from 78 to 67 mmHg.

This is to be contrasted with changes observed in Doppler hepatic venous flow after inhaled prostacyclin in a 55 years-old patient scheduled to undergo mitral valve and tricuspid valve repair. The Doppler hepatic venous flow had a systolic or S wave of negative value that became positive and less pronounced within 18 minutes following the administration prior to extra-corporal circulation of inhaled prostacyclin. This was associated with a reduction in mean pulmonary artery pressure from 36 to 29 mmHg, pulmonary vascular resistance index from 589 to 267 dynes.s/cm⁵/m² and an increase in cardiac index from 1.9 to 2.4 L/min/m². Heart rate and mean arterial pressure decreased respectively from 63 to 52 beats/min and from 76 to 64 mmHg. There were no difficult separation from bypass (cardiopulmonary bypass time of 138 min) and no vasoactive drugs used post-operatively.

Applications

Combining the above-described animal study, the above-described examples regarding human subjects and current knowledge of human physiology, for example with the above-described pathophysiological model of hemodynamic instability in cardiac surgical patients, we obtain a method for reducing the severity of an hemodynamic instability in a subject undergoing a cardiac surgery involving an extra-corporal circulation, the method including the administration through inhalation of a therapeutically effective amount of a vasodilatator to the subject.

The vasodilatator is administered at least in part prior to the extra-corporal circulation. In some embodiments of the invention, the vasodilatator is administered at least in part after anaesthesia of the subject. For example, and non-limitatively, the vasodilatator is started to be administered between about 10 minutes and about 30 minutes prior to the beginning of the extra-corporal circulation, and in some cases about 15 minutes prior to the beginning of the extra-corporal circulation.

The skilled medical practitioner will readily determine a suitable dosage for prostacyclin selected in an interval of about 0.1-100,000 μg. In some embodiments of the invention, the prostacyclin is administered in an amount of about 60-120 μg, and in some cases in an amount of about 90 μg.

In a specific embodiment of the invention, the prostacyclin is administered over a time interval of about 5-20 minutes, and in some cases over a time interval of about 10 minutes. The prostacyclin is administered only once, or the administration is repeated during, and in some cases, after the extra-corporal circulation.

Similarly, the skilled medical practitioner will readily determine a suitable dosage for milrinone selected in an interval of about 0.01-1000 mg. In some embodiments of the invention, the milrinone is administered in an amount of about 3-6 mg, and in some cases in an amount of about 0.05-1 mg/(kg body weight of the subject).

In a specific embodiment of the invention, the milrinone is administered over a time interval of about 5-20 minutes, and in some cases over a time interval of about 10 minutes. The milrinone is administered only once, or the administration is repeated during, and in some cases, after the extra-corporal circulation.

The above-described administration is non-limitatively suitable when the hemodynamic instability is associated with a dilatation of the right ventricle. In some cases, this dilatation of the right ventricle is a result of a pulmonary hypertension in the subject and the vasodilatator dilates blood vessels within the lungs of the subject while substantially not dilatating blood vessels outside of the lungs of the subject.

The above-described administration of a vasodilatator also gives a method for reducing the morbidity of a subject in cardiac surgeries involving an extra-corporal circulation and a method for facilitating weaning from extra-corporal circulation of a subject during a cardiac surgery.

The above-described administration of a vasodilatator also includes the use of an inhaled vasodilatator for reducing the severity of an hemodynamic instability in a subject undergoing a cardiac surgery involving an extra-corporal circulation, the use of an inhaled vasodilatator for reducing the morbidity of a subject in cardiac surgeries involving an extra-corporal circulation, and the use of an inhaled vasodilatator for facilitating weaning from extra-corporal circulation of a subject during a cardiac surgery.

The above-mentioned vasodilatators are used and administered either alone or in combination with one or more of these vasodilatators.

Although the present invention has been described hereinabove by way of preferred embodiments thereof, it can be modified without departing from the spirit, scope and nature of the subject invention, as defined in the appended claims.

TABLE 1
Summary of randomized controlled trials on milrinone in cardiac surgery

| Ref | n | Population | Dosage | Result |
|---|---|---|---|---|
| ⁷ | 32 | LVEF ≦ 35% | placebo vs bolus 50 ug/kg | All patients with milrinone |
| | | PCWP ≧ 20 mmHg | perfusion 0.5 ug/kg/min | wean from bypass vs 5/15 |
| | | pre- | | placebo |
| | | bypass | | |
| ¹² | 37 | Patients on | placebo vs bolus 50 ug/kg | Higher cardiac index and |
| | | cathecolamines | vs bolus 50 ug/kg and | velocity of shortening |
| | | post- | perfusion of 0.5 ug/kg/min | measured by TEE in all three |
| | | bypass | vs bolus 75 ug/kg and | milrinone group |
| | | | perfusion 0.75 ug/kg/min | |
| ¹³ | 44 | Elective | bolus of amrinone 0.75 mg/kg | amrinone and milrinone |
| | | cardiac | or milrinone 25 μg/kg | produced similar |
| | | surgery | | hemodynamic effects |
| ¹⁰ | 48 | patients with | (1) low pre-CPB | infusion of epinephrine in 5 of |
| | | a low pre- | CI/placebo, (2) low pre- | the 12 patients for |
| | | CPB cardiac | CPB CI/milrinone, (3) high | hemodynamic support vs |
| | | index (CI) | pre-CPB CI/placebo, and | nepinephrine in 6 of the 12 |
| | | <2.5 L/min/m2 | (4) high pre-CPB | patients in the low pre-CPB CI |
| | | and in | CI/milrinone | groups |
| | | patients with | milrinone 20 ug/kg and | |
| | | a high pre- | perfusion 0.2 ug/kg/min | |
| | | CPB CI (>or = 2.5 L/min/m²) | | |
| ⁹ | 45 | Pulmonary | Group 1 milrinone (n = 15) | Group 3 (40 ppm)higher right |
| | | hypertension | Group 2 20 ppm NO (n = 15) | ventricular ejection fraction |
| | | | Group 3 40 ppm NO (n = 15) | compared to group 1 and 2. |
| | | | | The milrinone group required |
| | | | | significantly more |
| | | | | phenylephrine in the intensive |
| | | | | care unit |
| ¹¹ | 120 | Low CO after | milrinone (M), 50 μg/kg and | group D had greater |
| | | cardiac | perfusion of 0.5 μg/kg/min vs | increases in cardiac index |
| | | surgery | dobutamine (D), 10 to 20 μg/kg/min | Group M had greater |
| | | | | decreases in mean pulmonary |
| | | | | capillary wedge pressure |
| | | | | Milrinone and dobutamine |
| | | | | both appropriate and |
| | | | | comparable |

TABLE 2
Clinical studies on inhaled prostacyclin

| | | | | | | Pulmonary | |
|---|---|---|---|---|---|---|---|
| Reference | Indication | Population | n | NO | Oxygenation | Hemodynamics | Side Effects |
| 19 | ARDS | ICU | 3 | | Improvement | Improvement | |
| 34 | PHT | Newborn | 2 | | Improvement | Improvement | |
| | | | | | | in ½ | |
| 35 | Pneumonia | Pneumonia | 12 | | Improvement | Selective | Shunt increase and |
| | | with or | | | in | improvement | hypotension in |
| | | without | | | non- | in non- | fibrosis group |
| | | fibrosis | | | fibrosis | fibrosis | |
| 36 | PHT | Infant in cath | 1 | | NA | Improvement | Improvement in CO |
| | | lab | | | | | |
| 37 | PHT | Newborn | 1 | Y | Improvement | Improvement | No comparison |
| | | post-bypass | | | | | with NO |
| 38 | ARDS | Children | 3 | Y | Improvement | Improvement | Hypotension with |
| | | | | | | | higher dosage in |
| | | | | | | | one child |
| | | | | | | | Equivalent effect to |
| | | | | | | | NO |
| 39 | ARDS + PHT | ICU patients | 8 | | Improvement | Improvement | Slight decrease in |
| | | | | | | | Cl |
| 40 | PHT | Acute | 1 | | Improvement | Improvement | |
| | | pulmonary | | | | | |
| | | embolism | | | | | |
| 21 | ARDS | ICU patients | 8 | Y | Improvement | Improvement | Hypotension at |
| | | | | | | | higher dosage, NO |
| | | | | | | | better for |
| | | | | | | | oxygenation |
| | | | | | | | PGl₂ better for |
| | | | | | | | pulmonary |
| | | | | | | | hemodynamics |
| 20 | PHT | Primary PHT | 6 | Y | Improvement | Improvement | Increase in CO |
| | | and CREST | | | | | PGl₂ better for |
| | | | | | | | oxygenation and |
| | | | | | | | pulmonary |
| | | | | | | | hemodynamics |
| 41 | PHT | Post cardiac | 9 | | No | Improvement | Improvement in RV |
| | | surgery | | | change | | function |
| 42 | ARDS | ICU patients | 5 | | Improvement | No change | |
| 43 | Severe | ARDS + amniotic | 2 | | Improvement | Improvement | |
| | hypoxia | fluid | | | | | |
| | | embolism | | | | | |
| 44 | PHT | Preterm | 1 | | Improvement | NA | |
| | | newborn | | | | | |
| 45 | PHT | Chronic PHT | 12 | Y | NA | Improvement | Increase in CO |
| | | | | | | | PGl₂ better for |
| | | | | | | | pulmonary |
| | | | | | | | hemodynamics |
| 25 | PHT | Heart | 10 | Y | Improvement | Improvement | Increase in CO |
| | | transplant | | | | | PGl₂ equivalent to |
| | | candidates | | | | | NO |
| 46 | PHT | Systemic | 1 | | Improvement | Improvement | |
| | | sclerosis | | | | | |
| 47 | PHT | NHYA III-IV | 18 | | NA | NA | Increase in expired |
| | | with PHT | | | | | NO in PHT only |
| 48 | PHT | Interstitial | 8 | Y | No | Improvement | Increase in CO and |
| | | lung fibrosis | | | change | | RVEF |
| | | | | | | | PGl₂ better for |
| | | | | | | | pulmonary |
| | | | | | | | hemodynamics |
| 22 | ARDS | ICU patients | 9 | | Improvement | No change | |
| 49 | PHT | lntraoperative | 5 | | NA | Improvement | Increase in CO |
| 50 | ARDS | ICU patients | 15 | | Mixed | No change | Pulmonary ARDS |
| | | | | | response | | did not respond as |
| | | | | | | | opposed to extra- |
| | | | | | | | pulmonary ARDS |
| 31 | ARDS + PHT | ICU and OR | 35 | Y | Improvement | Improvement | Hypotension and |
| | | patients | | | | | bronchospasm |
| | | | | | | | observed |
| 27 | PHT | Cardiac | 20 | | Improvement | Improvement | None |
| | | surgical | | | | | |
| | | patients | | | | | |
| 33 | PHT from | Cardiac | 1 | | Improvement | Improvement | None |
| | Carbon | surgical | | | | | |
| | dioxide | patients | | | | | |
| | embolism | | | | | | |
| 30 | PHT after | Cardiac | 1 | | Improvement | Improvement | |
| | valvular | surgical | | | | | |
| | surgery | patients | | | | | |

ARDS = adult respiratory distress syndrome,
PHT = pulmonary hypertension,
PVR = pulmonary vascular resistance,
P/S = ratio of pulmonary and systemic vascular resistance,
CO = cardiac output,
NO = nitric oxide
NA not reported,
ABG = arterial blood gases,
RVEF = right ventricular ejection fraction,
(A-a)O2 = alveolar to arterial gradient in oxygen.

TABLE 3
The amplitude of the contraction to KCl (60 mmol/L) and the
concentration of PE used to obtain 50% of contraction to KCl in
porcine subjects (see detailed description for complete
description of data).

| | Control | CEC | Mil INH | Mil IV |
|---|---|---|---|---|
| KCL | 5.1 +/− 0.3 | 3.9 +/− 2.2 | 4.8 +/− 0.28 | 4.0 +/− 0.3 |
| Dose PE | 5.5 +/− 0.9 | 7.0 +/− 9.6 | 8.3 +/− 0.93*,** | 4.1 +/− 0.3 |

*p < 0.01 vs intravenous milrinone
**p < 0.05 vs control

### LIST OF REFERENCES

1. Gage J, Rutman H, Lucido D, LeJemtel T H: Additive effects of dobutamine and amrinone on myocardial contractility and ventricular performance in patients with severe heart failure. Circulation 1986; 74: 367-73
2. Monrad E S, McKay R G, Baim D S, Colucci W S, Fifer M A, Heller G V, Royal H D, Grossman W: Improvement in indexes of diastolic performance in patients with congestive heart failure treated with milrinone. Circulation 1984; 70: 1030-7
3. Lobato E B, Florete O J, Bingham H L: A single dose of milrinone facilitates separation from cardiopulmonary bypass in patients with pre-existing left ventricular dysfunction. British Journal of Anaesthesia 1998; 81: 782-4
4. Lobato E B, Gravenstein N, Martin T D: Milrinone, not epinephrine, improves left ventricular compliance after cardiopulmonary bypass. J. Cardiothorac. Vasc. Anesth. 2000; 14: 374-7
5. Hardy J F, Searle N, Roy M, Perrault J: Amrinone, in combination with norepinephrine, is an effective first-line drug for difficult separation from cardiopulmonary bypass. Canadian Journal of Anaesthesia-Journal Canadien d'Anesthesie 1993; 40(6): 495-501
6. Sherry K M, Locke T J: Use of milrinone in cardiac surgical patients. Cardiovasc Drugs Ther 1993; 7: 671-5
7. Doolan L A, Jones E F, Kalman J, Buxton B F, Tonkin A M: A placebo-controlled trial verifying the efficacy of milrinone in weaning high-risk patients from cardiopulmonary bypass. Journal of Cardiothoracic and Vascular Anesthesia 1997; 11: 37-41
8. Kikura M, Levy J H, Bailey J M, Shanewise J S, Michelsen L G, Sadel S M: A bolus dose of 1.5 mg/kg amrinone effectively improves low cardiac output state following separation from cardiopulmonary bypass in cardiac surgical patients. Acta Anaesthesiologica Scandinavica 1998; 42: 825-33
9. Solina A, Papp D, Ginsberg S, Krause T, Grubb W, Scholz P, Pena L L, Cody R: A comparison of inhaled nitric oxide and milrinone for the treatment of pulmonary hypertension in adult cardiac surgery patients. J. Cardiothorac. Vasc. Anesth 2000; 14: 12-7
10. Yamada T, Takeda J, Katori N, Tsuzaki K, Ochiai R: Hemodynamic effects of milrinone during weaning from cardiopulmonary bypass: comparison of patients with a low and high prebypass cardiac index. J. Cardiothorac. Vasc. Anesth 2000; 14: 367-73
11. Feneck R O, Sherry K M, Withington P S, Oduro-Dominah A: Comparison of the hemodynamic effects of milrinone with dobutamine in patients after cardiac surgery. J. Cardiothorac. Vasc. Anesth 2001; 15: 306-15
12. Kikura M, Levy J H, Michelsen L G, Shanewise J S, Bailey J M, Sadel S M, Szlam F: The effect of milrinone on hemodynamics and left ventricular function after emergence from cardiopulmonary bypass. Anesthesia and Analgesia 1997; 85: 16-22
13. Rathmell J P, Prielipp R C, Butterworth J F, Williams E, Villamaria F, Testa L, Viscomi C, Iftleman F P, Baisden C E, Royster R L: A multicenter, randomized, blind comparison of amrinone with milrinone after elective cardiac surgery. Anesthesia and Analgesia 1998; 86: 683-90
14. Cuffe M S, Califf R M, Adams K F, Jr., Benza R, Bourge R, Colucci W S, Massie B M, O'Connor C M, Pina I, Quigg R, Silver M A, Gheorghiade M: Short-term intravenous milrinone for acute exacerbation of chronic heart failure: a randomized controlled trial. Journal of the American Medical Association 2002; 287: 1541-7
15. DiBianco R, Shabetai R, Kostuk W, Moran J, Schlant R C, Wright R: A comparison of oral milrinone, digoxin, and their combination in the treatment of patients with chronic heart failure. N. Engl. J. Med. 1989; 320: 677-83
16. Uretsky B F, Jessup M, Konstam M A, Dec G W, Leier C V, Benotti J, Murali S, Herrmann H C, Sandberg J A: Multicentertrial of oral enoximone in patients with moderate to moderately severe congestive heart failure. Lack of benefit compared with placebo. Enoximone Multicenter Trial Group. Circulation 1990; 82: 774-80
17. Haraldsson s A, Kieler-Jensen N, Ricksten S E: The additive pulmonary vasodilatory effects of inhaled prostacyclin and inhaled milrinone in postcardiac surgical patients with pulmonary hypertension. Anesth. Analg. 2001; 93:
18. Scheeren T, Radermacher P: Prostacyclin (PGI2): new aspects of an old substance in the treatment of critically ill patients. Intensive Care Med. 1997; 23: 146-58
19. Walmrath D, Schneider T, Pilch J, Grimminger F, Seeger W: Aerosolised prostacyclin in adult respiratory distress syndrome. Lancet 1993; 342: 961-2
20. Olschewski H, Walmrath D, Schermuly R, Ghofrani A, Grimminger F, Seeger W: Aerosolized prostacyclin and iloprost in severe pulmonary hypertension. Ann. Intern. Med. 1996; 124: 820-4
21. Zwissler B, Kemming G, Habler O, Kleen M, Merkel M, Haller M, Briegel J, Welte M, Peter K: Inhaled prostacyclin (PGI2) versus inhaled nitric oxide in adult respiratory distress syndrome. Am. J. Respir. Crit Care Med. 1996; 154: 1671-7
22. van Heerden P V, Barden A, Michalopoulos N, Bulsara M K, Roberts B L: Dose-response to inhaled aerosolized prostacyclin for hypoxemia due to ARDS. Chest 2000; 117: 819-27
23. Montalescot G, Drobinski G, Meurin P, Maclouf J, Sotirov I, Philippe F, Choussat R, Morin E, Thomas D: Effects of prostacyclin on the pulmonary vascular tone and cardiac contractility of patients with pulmonary hypertension secondary to end-stage heart failure. Am. J. Cardiol. 1998; 82: 749-55
24. Rich S, McLaughlin W: The effects of chronic prostacyclin therapy on cardiac output and symptoms in primary pulmonary hypertension. J. Am. Coll. Cardiol. 1999; 34:1184-7
25. Haraldsson A, Kieler-Jensen N, Nathorst-Wesffelt U, Bergh C H, Ricksten S E: Comparison of inhaled nitric oxide and inhaled aerosolized prostacyclin in the evaluation of heart transplant candidates with elevated pulmonary vascular resistance. Chest 1998; 114: 780-6
26. Haraldsson A, Kieler-Jensen N, Wadenvik H, Ricksten S E: Inhaled prostacyclin and platelet function after cardiac surgery and cardiopulmonary bypass. Intensive Care Med. 2000; 26: 188-94
27. Hache M, Denault A, Belisle S, Robitaille D, Couture P, Sheridan P, Pellerin M, Babin D, Noel N, Guertin MC, Martineau R, Dupuis J: Inhaled epoprostenol (prostacyclin) and pulmonary hypertension before cardiac surgery. J. Thorac. Cardiovasc. Surg. 2003; 125: 642-9
28. Habler o, Kleen M, Zwissler B, Pusch R, Welte M, Vogelmeier C, Kempter B, Krombach F, Messmer K: Inhalation of prostacyclin (PGI2) for 8 hours does not produce signs of acute pulmonary toxicity in healthy lambs. Intensive Care Med. 1996; 22: 426-33
29. Long W A, Rubin L J: Prostacyclin and PGE1 treatment of pulmonary hypertension. Am. Rev. Respir. Dis. 1987; 136: 773-6
30. Lowson S M, Doctor A, Walsh B K, Doorley P A: Inhaled prostacyclin for the treatment of pulmonary hypertension after cardiac surgery. Crit Care Med. 2002; 30: 27624
31. Hache M, Denault A Y, Belisle S, Couture P, Babin D, Tetrault F, Guimond J G: Inhaled prostacyclin (PGI(2)) is an effective addition to the treatment of pulmonary hypertension and hypoxia in the operating room and intensive care unit: [L'inhalation de prostacycline (PGI(2)) est un traitement complementaire efficace de l'hypertension pulmonaire et de l'hypoxie observees en salle d'operation et a l'unite des soins intensifs]. Can. J. Anaesth. 2001; 48: 924-9
32. Weinberger B, Laskin D L, Heck DE, Laskin J D: The toxicology of inhaled nitric oxide. Toxicol. Sci. 2001; 59: 5-16
33. Martineau A, Arcand G, Couture P, Babin D, Perreault L P, Denault A: Transesophageal echocardiographic diagnosis of carbon dioxide embolism during minimally invasive saphenous vein harvesting and treatment with inhaled epoprostenol. Anesth. Analg. 2003; 96: 962-4, table
34. Bindl L, Fahnenstich H, Peukert U: Aerosolised prostacyclin for pulmonary hypertension in neonates. Arch. Dis. Child Fetal Neonatal Ed 1994; 71: F214-F216
35. Walmrath D, Schneider T, Pilch J, Schermuly R, Grimminger F, Seeger W: Effects of aerosolized prostacyclin in severe pneumonia. Impact of fibrosis. Am. J. Respir. Crit Care Med. 1995; 151: 724-30
36. Santak B, Schreiber M, Kuen P, Lang D, Radermacher P: Prostacyclin aerosol in an infant with pulmonary hypertension. Eur. J. Pediatr. 1995; 154: 233-5
37. Zwissler B, Rank N, Jaenicke U, Schurle B, Welte M, Reichart B, Netz H, Messmer K, Peter K: Selective pulmonary vasodilation by inhaled prostacyclin in a newborn with congenital heart disease and cardiopulmonary bypass. Anesthesiology 1995; 82: 1512-6
38. Pappert D, Busch T, Gerlach H, Lewandowski K, Radermacher P, Rossaint R: Aerosolized prostacyclin versus inhaled nitric oxide in children with severe acute respiratory distress syndrome. Anesthesiology 1995; 82: 1507-11
39. Bein T, Metz C, Keyl C, Sendtner E, Pfeifer M: Cardiovascular and pulmonary effects of aerosolized prostacyclin administration in severe respiratory failure using a ventilator nebulization system. J. Cardiovasc. Pharmacol. 1996; 27: 583-6
40. Webb SA, Stott S, van Heerden PV: The use of inhaled aerosolized prostacyclin (IAP) in the treatment of pulmonary hypertension secondary to pulmonary embolism. Intensive Care Med. 1996; 22: 353-5
41. Haraldsson A, Kieler-Jensen N, Ricksten S E: Inhaled prostacyclin for treatment of pulmonary hypertension after cardiac surgery or heart transplantation: a pharmacodynamic study. J. Cardiothorac. Vasc. Anesth 1996; 10: 864-8
42. van Heerden P V, Blythe D, Webb S A: Inhaled aerosolized prostacyclin and nitric oxide as selective pulmonary vasodilators in ARDS—a pilot study. Anaesth. Intensive Care 1996; 24: 564-8
43. van Heerden P V, Webb S A, Hee G, Corkeron M, Thompson WR: Inhaled aerosolized prostacyclin as a selective pulmonary vasodilator for the treatment of severe hypoxaemia. Anaesth. Intensive Care 1996; 24: 87-90
44. Soditt V, Aring C, Groneck P: Improvement of oxygenation induced by aerosolized prostacyclin in a preterm infant with persistent pulmonary hypertension of the newborn. Intensive Care Med. 1997; 23: 1275-8
45. Mikhail G, Gibbs J, Richardson M, Wright G, Khaghani A, Banner N, Yacoub M: An evaluation of nebulized prostacyclin in patients with primary and secondary pulmonary hypertension. Eur. Heart J. 1997; 18: 1499-504
46. Parameswaran K, Purcell I, Farrer M, Holland C, Taylor I K, Keaney N P: Acute effects of nebulised epoprostenol in pulmonary hypertension due to systemic sclerosis. Respir. Med. 1999; 93: 75-8
47. Forrest I A, Small T, Corris P A: Effect of nebulized epoprostenol (prostacyclin) on exhaled nitric oxide in patients with pulmonary hypertension due to congenital heart disease and in normal controls. Clin. Sci. (Colch.) 1999; 97: 99-102
48. Olschewski H, Ghofrani H A, Walmrath D, Schermuly R, Temmesfeld-Wollbruck B, Grimminger F, Seeger W: Inhaled prostacyclin and iloprost in severe pulmonary hypertension secondary to lung fibrosis. Am. J. Respir. Crit Care Med. 1999; 160: 600-7
49. Schroeder R A, Wood G L, Plotkin J S, Kuo P C: Intraoperative use of inhaled PGI(2) for acute pulmonary hypertension and right ventricular failure. Anesthesia and Analgesia 2000; 91: 291-5
50. Domenighefti G, Stricker H, Waldispuehl B: Nebulized prostacyclin (PGI2) in acute respiratory distress syndrome: impact of primary (pulmonary injury) and secondary (extrapulmonary injury) disease on gas exchange response. Crit Care Med. 2001; 29: 57-62
51. Costachescu, T., Denault, A. Y., Guimond, J. G., et al. 2002, The hemodynamically unstable patient in the intensive care unit: Hemodynamic vs. transesophageal echocardiographic monitoring, Crit. Care Med. 30, 1214-1223.
52. Demers, P., Elkouri, S., Martineau, R., et al. 2000, Outcome with high blood lactate levels during cardiopulmonary bypass in adult cardiac operation, Ann. Thorac. Surg. 70, 2082-2086.
53. Bernard, F., Denault, A., Babin, D., et al. 2001, Diastolic dysfunction is predictive of difficult weaning from cardiopulmonary bypass, Anesth. Analg. 92, 291-298.
54. Salem, R., Denault, A. Y., Couture, P., et al. 2003, Diastolic dysfunction as an independent predictor of mortality in cardiac surgery, Can. J. Anaesth. (abstract presented at the 2003 Canadian Society meeting) (http://www.cja-jca.org/cqi/content/full/50/suppl 1/A95).
55. Fortier S, DeMaria R G, Lamarche Y, Malo O, Denault A, Desjardins F, Carrier M, Perrault L P, Inhaled prostacyclin reduces cardiopulmonary bypass-induced pulmonary endothelial dysfunction via increased cyclic adenosine monophosphate levels. J Thorac Cardiovasc Surg. 2004 July;128(1):109-16.
56. Hache, M., Denault, A., Belisle, S., et al. 2003, Inhaled epoprostenol (prostacyclin) and pulmonary hypertension before cardiac surgery, J. Thorac. Cardiovasc. Surg. 125, 642-649
57. Hache, M., Denault, A. Y., Belisle, S., et al. 2001, Inhaled prostacyclin (PGI(2)) is an effective addition to the treatment of pulmonary hypertension and hypoxia in the operating room and intensive care unit: [L'inhalation de prostacycline (PGI(2)) est un traitement complémentaire efficace de l'hypertension pulmonaire et de l'hypoxie observées en salle d'opération et à l'unité des soins intensifs], Can. J. Anaesth. 48, 924-929.
58. Robitaille, A., Denault, A., Couture, P., et al. 2003, The mean arterial to pulmonary artery pressure ratio in cardiac surgical patients is a predictor of adverse hemodynamic outcome in the intensive care unit, Can. J. Anaesth. (abstract presented at the 2003 Canadian Society meeting) (http://www.cja-jca.org/cqi/content/full/50/suppl_{—}1/A69).
59. Costachescu, T., Denault, A. Y., Guimond, J. G., et al. 2002, The hemodynamically unstable patient in the intensive care unit: Hemodynamic vs. transesophageal echocardiographic monitoring, Crit. Care Med. 30, 1214-1223.
60. Malouf, J. F., Enriquez-Sarano, M., Pellikka, P. A., et al. 2002, Severe pulmonary hypertension in patients with severe aortic valve stenosis: clinical profile and prognostic implications, J. Am. Coll. Cardiol. 40, 789-795.
61. Higgins, T. L., Estafanous, F. G., Loop, F. D., et al. 1992, Stratification of morbidity and mortality outcome by preoperative risk factors in coronary artery bypass patients. A clinical severity score, JAMA 267, 2344-2348.
62. Bossone, E., Paciocco, G., larussi, D., et al. 2002, The prognostic role of the ECG in primary pulmonary hypertension, Chest 121, 513-518.
63. Marcus, J. T., Vonk, N. A., Roeleveld, R. J., et al. 2001, Impaired left ventricular filling due to right ventricular pressure overload in primary pulmonary hypertension: noninvasive monitoring using MRI, Chest 119, 1761-1765.
64. Haraldsson A, Kieler-Jensen N, Ricksen S E. The additive pulmonary vasodilatory effects of inhaled prostacyclin and inhaled milrinone in postcardiac surgery patients with pulmonary hypertension. Anesth Analg 2001; 93: 1439-45.
65. Morita K, Ihnken K, Buckberg G D, Sherman M P, Ignarro L J. Pulmonary vasoconstriction due to impaired nitric oxide production after cardiopulmonary bypass. Ann Thorac Surg. 1996;61:1775-80.
66. Niwano K, Arai M, Tomaru K et al. Transcriptionnal stimulation of the eNOS gen by the stable prostacyclin analogue Beraprost is mediated through cAMP-responsive element in vascular endothelial cells. Circ Res. 2003; 93: 523-530.
67. Lockinger A, Schutte H, Walmrath D, Seeger W, Grimminger F. Protection against gas exchange abnormalities by pre-aerosolized PGE1, iloprost and nitroprusside in lung ischemia-reperfusion. Transplantation. 2001 Jan. 27;71 (2):185-93.

1. A method for reducing the severity of an hemodynamic instability in a subject undergoing a cardiac surgery involving an extra-corporal circulation, said method comprising the administration through inhalation of a therapeutically effective amount of a vasodilatator to the subject.

2. A method as defined in claim 1, wherein the vasodilatator is administered at least in part prior to the extra-corporal circulation.

3. A method as defined in claim 2, wherein the vasodilatator is administered at least in part after anaesthesia of the subject

4. A method as defined in claim 3, wherein the vasodilatator is started to be administered between about 10 minutes and about 30 minutes prior to the beginning of the extra-corporal circulation.

5. A method as defined in claim 4, wherein the vasodilatator is started to be administered about 15 minutes prior to the beginning of the extra-corporal circulation.

6. A method as defined in claim 4, wherein the vasodilatator is selected from the group consisting of: prostacyclin (flolan®), amrinone (inocor®), dobutamine (dobutrex®), nitroglycerine, nitroprussiate (nipruss®) and milrinone (primacor®).

7. A method as defined in claim 6, wherein the vasodilatator is prostacyclin.

8. A method as defined in claim 7, wherein the prostacyclin is administered in an amount of about 0.1-100,000 μg.

9. A method as defined in claim 8, wherein the prostacyclin is administered in an amount of about 60-120 μg.

10. A method as defined in claim 9, wherein the prostacyclin is administered in an amount of about 90 μg.

11. A method as defined in claim 9, wherein the prostacyclin is administered over a time interval of about 5-20 minutes.

12. A method as defined in claim 11, wherein the prostacyclin is administered over a time interval of about 10 minutes.

13. A method as defined in claim 6, wherein the vasodilatator is milrinone.

14. A method as defined in claim 13, wherein the milrinone is administered in an amount of about 0.01-1000 mg.

15. A method as defined in claim 14, wherein the milrinone is administered in an amount of about 3-6 mg.

16. A method as defined in claim 14, wherein the milrinone is administered in an amount of about 0.05-1 mg/(kg body weight of the subject).

17. A method as defined in claim 9, wherein the milrinone is administered over a time interval of about 5-20 minutes.

18. A method as defined in claim 11, wherein the milrinone is administered over a time interval of about 10 minutes.

19. A method as defined in claim 1 wherein the hemodynamic instability is associated with a dilatation of the right ventricle.

20. A method as defined in claim 19, wherein the dilatation of the right ventricle is a result of a pulmonary hypertension in the subject.

21. A method as defined in claim 1, wherein the vasodilatator dilates blood vessels within the lungs of the subject.

22. A method as defined in claim 21, wherein the vasodilatator dilates blood vessels within the lungs of the subject, while substantially not dilatating blood vessels outside of the lungs of the subject.

23. A method as defined in claim 1, wherein the subject is a mammal.

24. A method as defined in claim 23, wherein the mammal is human.

25. A method for reducing the morbidity of a subject in cardiac surgeries involving an extra-corporal circulation, said method comprising the administration through inhalation of a therapeutically effective amount of a vasodilatator to the subject.

26. A method as defined in claim 25, wherein the vasodilatator is administered at least in part prior to the extra-corporal circulation.

27. A method as defined in claim 26, wherein the vasodilatator is administered at least in part after anaesthesia of the subject

28. A method as defined in claim 27, wherein the vasodilatator is started to be administered between about 10 minutes and about 30 minutes prior to the beginning of the extra-corporal circulation.

29. A method as defined in claim 28, wherein the vasodilatator is started to be administered about 15 minutes prior to the beginning of the extra-corporal circulation.

30. A method as defined in claim 28, wherein the vasodilatator is selected from the group consisting of: prostacyclin (flolan®), amrinone (inocor®), dobutamine (dobutrex®), nitroglycerine, nitroprussiate (nipruss®) and milrinone (primacor®).

31. A method as defined in claim 30, wherein the vasodilatator is prostacyclin.

32. A method as defined in claim 31, wherein the prostacyclin is administered in an amount of about 0.1-100,000 μg.

33. A method as defined in claim 32, wherein the prostacyclin is administered in an amount of about 60-120 μg.

34. A method as defined in claim 33, wherein the prostacyclin is administered in an amount of about 90 μg.

35. A method as defined in claim 33, wherein the prostacyclin is administered over a time interval of about 5-20 minutes.

36. A method as defined in claim 35, wherein the prostacyclin is administered over a time interval of about 10 minutes.

37. A method as defined in claim 30, wherein the vasodilatator is milrinone.

38. A method as defined in claim 37, wherein the milrinone is administered in an amount of about 0.01-1000 mg.

39. A method as defined in claim 38, wherein the milrinone is administered in an amount of about 3-6 mg.

40. A method as defined in claim 38, wherein the milrinone is administered in an amount of about 0.05-1 mg/(kg body weight of the subject).

41. A method as defined in claim 39, wherein the milrinone is administered over a time interval of about 5-20 minutes.

42. A method as defined in claim 41, wherein the milrinone is administered over a time interval of about 10 minutes.

43. A method as defined in claim 25 wherein the reducing of the morbidity of the subject is realized at least in part by reducing an hemodynamic instability associated with a dilatation of the right ventricle of the subject.

44. A method as defined in claim 43, wherein the hemodynamic instability is reduced by dilating blood vessels within the lungs of the subject.

45. A method as defined in claim 44, wherein the vasodilatator does not substantially dilatate blood vessels outside of the lungs of the subject.

46. A method as defined in claim 25, wherein the subject is a mammal.

47. A method as defined in claim 46, wherein the mammal is human.

48. A method for facilitating weaning from extra-corporal circulation of a subject during a cardiac surgery, said method comprising the administration through inhalation of a therapeutically effective amount of a vasodilatator.

49. A method as defined in claim 48, wherein the vasodilatator is administered at least in part prior to the extra-corporal circulation.

50. A method as defined in claim 49, wherein the vasodilatator is administered at least in part after anaesthesia of the subject

51. A method as defined in claim 50, wherein the vasodilatator is started to be administered between about 10 minutes and about 30 minutes prior to the beginning of the extra-corporal circulation.

52. A method as defined in claim 51, wherein the vasodilatator is started to be administered about 15 minutes prior to the beginning of the extra-corporal circulation.

53. A method as defined in claim 52, wherein the vasodilatator is selected from the group consisting of: prostacyclin (flolan®), amrinone (inocor®), dobutamine (dobutrex®), nitroglycerine, nitroprussiate (nipruss®) and milrinone (primacor®).

54. A method as defined in claim 53, wherein the vasodilatator is prostacyclin.

55. A method as defined in claim 54, wherein the prostacyclin is administered in an amount of about 0.1-100,000 μg.

56. A method as defined in claim 55, wherein the prostacyclin is administered in an amount of about 60-120 μg.

57. A method as defined in claim 56, wherein the prostacyclin is administered in an amount of about 90 μg.

58. A method as defined in claim 56, wherein the prostacyclin is administered over a time interval of about 5-20 minutes.

59. A method as defined in claim 11, wherein the prostacyclin is administered over a time interval of about 10 minutes.

60. A method as defined in claim 53, wherein the vasodilatator is milrinone.

61. A method as defined in claim 60, wherein the milrinone is administered in an amount of about 0.01-1000 mg.

62. A method as defined in claim 61, wherein the milrinone is administered in an amount of about 3-6 mg.

63. A method as defined in claim 61, wherein the milrinone is administered in an amount of about 0.05-1 mg/(kg body weight of the subject).

64. A method as defined in claim 62, wherein the milrinone is administered over a time interval of about 5-20 minutes.

65. A method as defined in claim 64, wherein the milrinone is administered over a time interval of about 10 minutes.

66. A method as defined in claim 48, wherein facilitating weaning from extra-corporal circulation includes reducing the dilatation of the right ventricle of the subject.

67. A method as defined in claim 48, wherein the vasodilatator dilates blood vessels within the lungs of the subject while substantially not dilatating blood vessels outside of the lungs of the subject.

68. A method as defined in claim 48, wherein the subject is a mammal.

69. A method as defined in claim 68, wherein the mammal is human.
